(19) United States
(12) Patent Application Publication
(10) Pub. No.: US 2017/0100158 A1
(43) Pub. Date: Apr. 13, 2017

(54) SURGICAL TREATMENT OF SHOULDER JOINT

(71) Applicant: OLYMPUS CORPORATION, Tokyo (JP)

(72) Inventors: Sohei UEDA, Tokyo (JP); Chie ONUMA, Tama-shi (JP); Manabu ISHIKAWA, Hachioji-shi (JP)

(21) Appl. No.: 14/878,764

(22) Filed: Oct. 8, 2015

(51) Int. Cl. A61B 17/32

(52) U.S. Cl. CPC A61B 17/320068; A61B 17/320016; A61B 90/361

(57) ABSTRACT

A surgical treatment of a shoulder joint, includes inserting an arthroscope and an ultrasonic device into the shoulder joint, and removing a bursa and removing a bone spur present under an acromion in a state seen with the arthroscope by use of the ultrasonic device which ultrasonically vibrates.

### BACKGROUND OF THE INVENTION

1. Field of the Invention

The present invention relates to a surgical treatment of a shoulder joint which is to be performed under an arthroscope.

2. Description of the Related Art

In recent years, there have been performed arthroscopic surgical treatments for various joints of knees, feet, shoulders, elbows and the like. In such arthroscopic surgical treatments, various mechanical cutting tools such as a shaver and a burr have been utilized. In general, the shaver or a high frequency device is used to excise an unnecessary or damaged soft tissue, and the burr is used for a harder tissue such as a bone. Therefore, the treatment is performed while inserting and removing an optimum mechanical cutting tool through a joint cavity in accordance with a treatment object.

### BRIEF SUMMARY OF THE INVENTION

A surgical treatment of a shoulder joint of one embodiment according to the present invention comprises inserting an arthroscope and an ultrasonic device into the shoulder joint, and removing a bursa and removing a bone spur present under an acromion in a state seen with the arthroscope by use of the ultrasonic device which ultrasonically vibrates.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1A is a schematic view showing a treatment system for use in a surgical treatment of a shoulder joint;

FIG. 1B is a schematic view showing a part of the treatment system of FIG. 1A in detail;

FIG. 2 is a side view showing an ultrasonic probe of a treatment tool of the treatment system shown in FIG. 1B;

FIG. 3 is a top view of an ultrasonic probe shown in FIG. 2;

FIG. 4 is a schematic view showing a patient fixed at a beach chair position;

FIG. 5 is a schematic view of positions of a skeleton around a right shoulder joint and a scapula upper arm joint which are seen from the front;

FIG. 6 is a perspective view of an anatomical structure of the scapula upper arm joint which is seen from the front;

FIG. 7 is a schematic view showing each portal to access the scapula upper arm joint from a head part direction of the patient;

FIG. 8 is a perspective view showing a step of debriding a torn labrum from a glenoid by use of the ultrasonic probe in Bankart repair;

FIG. 9 is a perspective view showing a step of peeling the labrum from the glenoid by use of the ultrasonic probe in the Bankart repair;

FIG. 10A is a schematic view showing a state where the labrum is sutured at a correct position by use of anchors and sutures in a labrum ligament complex;

FIG. 10B is a cross-sectional view schematically showing the labrum completely peeled from the glenoid;

FIG. 10C is a cross-sectional view schematically showing a state where an anchor of the anchors and sutures is fixed to a hard tissue of the glenoid;

FIG. 10D is a cross-sectional view schematically showing a state where the suturing of the labrum is completed by using the anchors and sutures;

FIG. 11 is a perspective view showing a treatment of cleaning a position (a footprint region) of a root of a subscapular muscle tendon with the ultrasonic probe in repair of the subscapular muscle tendon in shoulder rotator cuff repair;

FIG. 12A is a perspective view showing a state where the subscapular muscle tendon is fixed to a humerus by the anchors and sutures in the repair of the subscapular muscle tendon;

FIG. 12B is a cross-sectional view schematically showing a state where the subscapular muscle tendon is fixed to the humerus by the anchors and sutures in the repair;

FIG. 13 is a front view showing the anatomical structure around a scapula upper arm joint;

FIG. 14 is a perspective view showing an affected area in which a cuff is torn;

FIG. 15 is a side view showing a treatment of debriding the torn cuff shown in FIG. 14 by use of the ultrasonic probe;

FIG. 16 is a perspective view showing a treatment of removing a subacromial bone spur in the shoulder rotator cuff repair;

FIG. 17 is a schematic view showing a state where the anchor is fixed to the humerus and then the cuff is being fixed by using the sutures in the shoulder rotator cuff repair; and

FIG. 18 is a schematic view showing a state where the cuff is fixed with the sutures in the shoulder rotator cuff repair shown in FIG. 17.

### DETAILED DESCRIPTION OF THE INVENTION

Embodiments of this invention will be described with

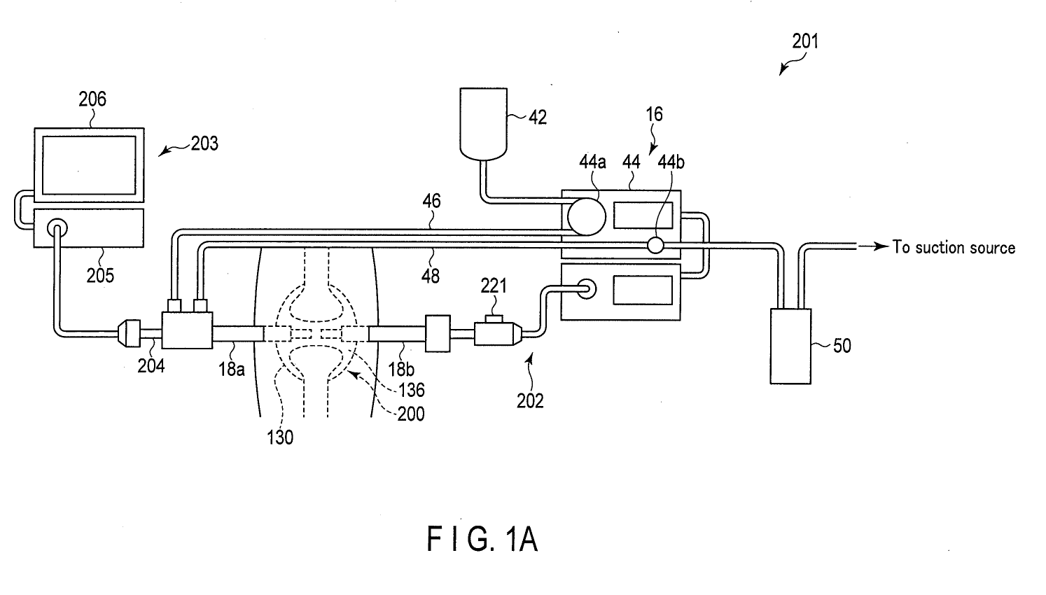

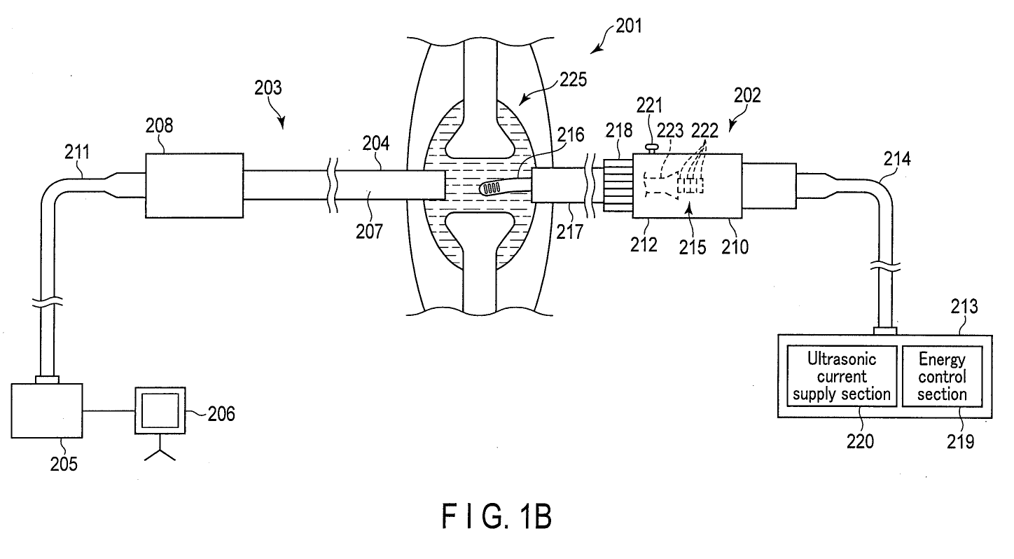

When a shoulder joint 200 is treated, for example, a treatment system 201 shown in FIG. 1A and FIG. 1B is used. The treatment system 201 has an arthroscope device 203, a treatment device 202, and a perfusion device 16.

The arthroscope device 203 has an arthroscope 204 to observe the inside of the shoulder joint 200, i.e., the inside of a joint cavity 136 of a patient, an arthroscope controller (an image processing unit) 205 that performs image processing on the basis of a subject image imaged by the arthroscope 204, and a monitor 206 that reflects the image generated by the image processing in the arthroscope controller 205.

The arthroscope 204 comprises an inserting portion 207 and a holding portion 208. In a treatment in which this arthroscope is used, a distal end of the inserting portion 207 is inserted into the shoulder joint 200. The holding portion 208 is connected to one end of a universal cord 211. The other end of the universal cord 211 is connected to the image processing unit 205, i.e., an image processor or the like. The image processing unit 205 is electrically connected to the display unit 206, i.e., the monitor or the like.

At the distal end of the inserting portion 207, an imaging element is disposed, and the imaging element is electrically connected to the image processing unit 205. The image acquired by the imaging element is processed by the image processing unit 205 and displayed in the display unit 206. It is to be noted that the arthroscope 204 is connected to an unshown light source unit, and a subject is irradiated with light emitted from the light source unit.

The perfusion device 16 includes a bag-shaped liquid source 42 that contains a perfusion liquid such as saline, a perfusion pump unit 44, a liquid supply tube 46 whose one end is connected to the liquid source 42, a liquid discharge tube 48, and a suction bottle 50 connected to one end of the liquid discharge tube 48. The suction bottle 50 is connected to a suction source attached to a wall of an operating room. In the perfusion pump unit 44, the perfusion liquid can be supplied from the liquid source 42 by a liquid supply pump 44a. Additionally, in the perfusion pump unit 44, suction/suction stop of the perfusion liquid in the joint cavity 136 of the shoulder joint 200 to the suction bottle 50 can be switched by opening/closing a pinching valve 44b as a liquid discharge valve.

The other end of the liquid supply tube 46 that is a liquid supply tube path is connected to a first cannula 18a. In consequence, the perfusion liquid can be supplied into the joint cavity 136 of the joint 200 via the first cannula 18a. The other end of the liquid discharge tube 48 that is a liquid discharge tube path is connected to the first cannula 18a. In consequence, the perfusion liquid can be discharged from the joint cavity 136 of the joint 200 via the first cannula 18a. It is to be noted that, needless to say, the other end of the liquid discharge tube 48 may be connected to a second cannula 18b, so that the perfusion liquid can be discharged from the joint 200.

It is to be noted that, here, the perfusion liquid can be supplied and discharged through the first cannula 18a, but a function that is capable of supplying and/or discharging the perfusion liquid may be imparted to, for example, the arthroscope 204. Similarly, the function that is capable of supplying and/or discharging the perfusion liquid may be imparted to a hand piece 212. In addition, a function that is capable of supplying and discharging the perfusion liquid through the second cannula 18b may be imparted. Furthermore, the perfusion liquid may be supplied and discharged from separate portals.

As shown in FIG. 1B, the treatment device 202 comprises the hand piece 212 as an ultrasonic device, a power source unit 213, and a cable 214 connecting the hand piece 212 to the power source unit 213. The treatment device 202 is one example of the ultrasonic device. The power source unit 213 has an energy control section 219, and an ultrasonic current supply section 220 to be controlled by the energy control section 219, thereby supplying power to a vibration generating section 215.

The hand piece 212 comprises a housing 210 constituting an outer shell, the vibration generating section 215 housed in the housing 210, a rod-like ultrasonic probe 216 connected to the vibration generating section 215, a hollow (cylindrical) sheath 217 that covers a periphery of the ultrasonic probe 216 to protect the ultrasonic probe 216, a knob 218 rotatably attached to the housing 210, and an energy input button (switch) 221 disposed in the housing 210.

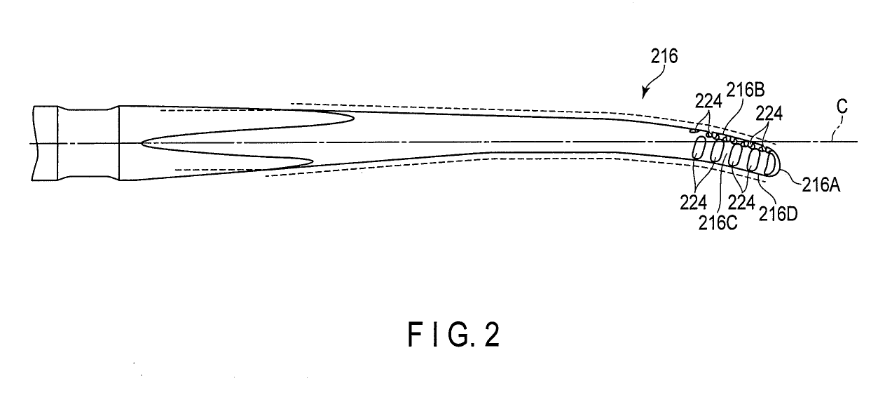

The housing 210 is connected to one end of the cable 214. The other end of the cable 214 is connected to the power source unit 213. The knob 218 is fixed to, for example, the ultrasonic probe 216, and the knob 218 is rotated to the housing 210, so that the ultrasonic probe 216 can be rotated around a central axis C (see FIG. 2). In addition, when an operator operates the energy input button 221, the energy control section 219 senses an operation input of the energy input button 221. Further, the energy control section 219 controls the ultrasonic current supply section 220 to supply power to the vibration generating section 215. Consequently, an ultrasonic vibration (ultrasonic energy) is transmitted to the ultrasonic probe 216, and the ultrasonic vibration can be imparted to a bone tissue (a biological tissue) of a treatment object via the ultrasonic probe 216. In consequence, it is possible to perform excision, removal, debridement and the like of the biological tissue.

It is to be noted that the energy input buttons 221 may be disposed. An amplitude of an ultrasonic vibrator can suitably be set by the energy control section 219. In consequence, by the operation of the energy input button 221, a frequency of the ultrasonic vibration to be output from the after-mentioned ultrasonic vibrator is the same, but the amplitude may be different. Therefore, the energy input button 221 can suitably switch the amplitude of the ultrasonic vibrator to states such as two large and small states. For example, when the amplitude can be switched to the two large and small states, the ultrasonic vibration of the small amplitude is for use in treating a comparatively soft tissue such as a synovial membrane or a bursa. The ultrasonic vibration of the large amplitude is for use in treating a comparatively hard tissue such as a bone (a bone spur 251).

It is to be noted that, for example, the two energy input buttons 221 may be disposed in parallel, or a hand switch and a foot switch may selectively be used. Additionally, when the one switch 221 is switched to be used, the ultrasonic vibration of the small amplitude may be output by one operation, and the ultrasonic vibration of the large amplitude may be output by two quick pressing operations as in a double click operation of a mouse for a computer.

The vibration generating section 215 comprises piezoelectric elements 222 and a horn member 223. The piezoelectric elements 222 receives the power supplied from the power source unit 213 to generate the ultrasonic vibration. The horn member 223 transmits the ultrasonic vibration to the ultrasonic probe 216 while enlarging the amplitude of the ultrasonic vibration generated by the piezoelectric elements 222.

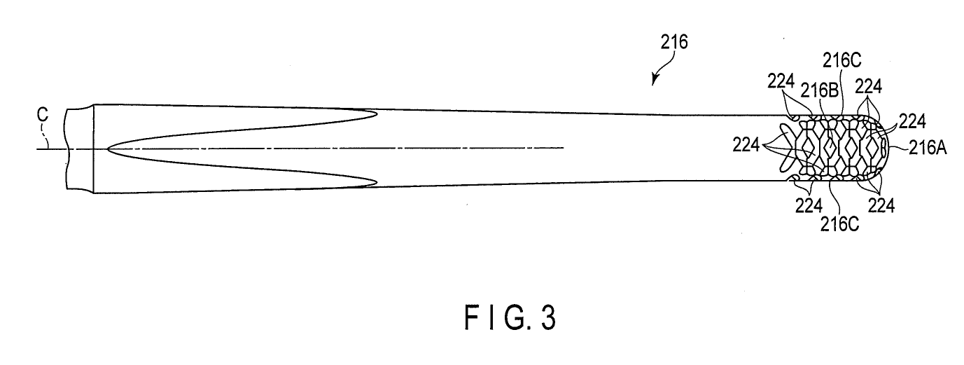

As shown in FIG. 2 and FIG. 3, the ultrasonic probe 216 is made of, for example, a metal material (e.g., a titanium alloy or the like) having a biocompatibility and is shaped in the form of a rod. The ultrasonic probe 216 has cutting blades 224 (treating portions) on a distal surface 216A, an upper surface 216B and both side surfaces 216C. The cutting blades 224 (the treating portions) are concentrated on a distal side of the ultrasonic probe 216, and abut on the biological tissue when the biological tissue is treated. The ultrasonic probe 216 is extended toward a back surface 216D side so that an upper surface 216B side is projected.

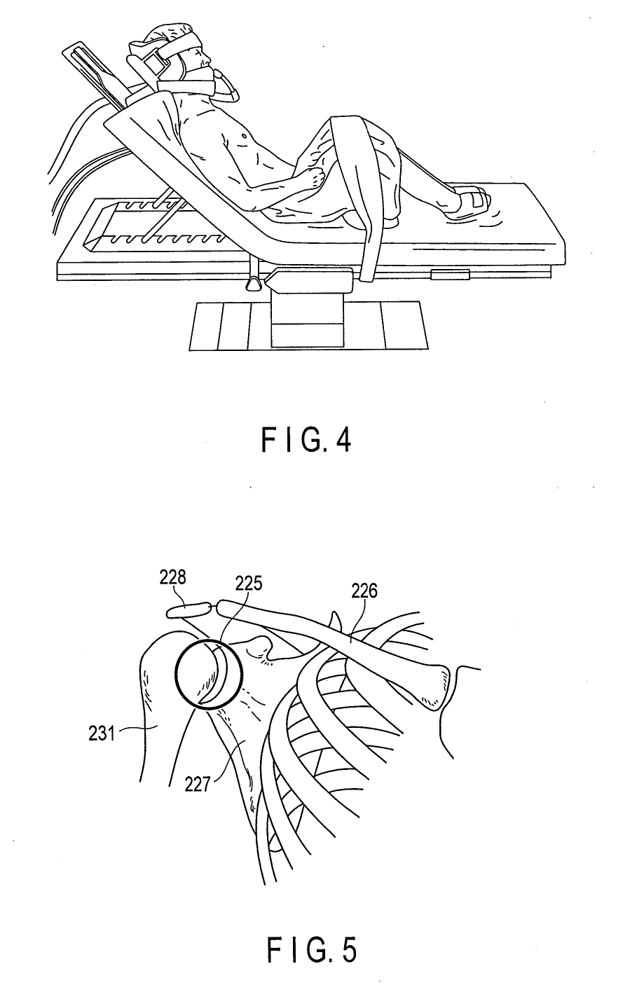

Next, Bankart repair to be performed under the arthroscope will be described with reference to FIG. 4 to FIG. 10. The Bankart repair is performed for the purpose of treating recurrent shoulder joint dislocation. The Bankart repair is carried out while circulating the perfusion liquid through the shoulder joint by a well-known method.

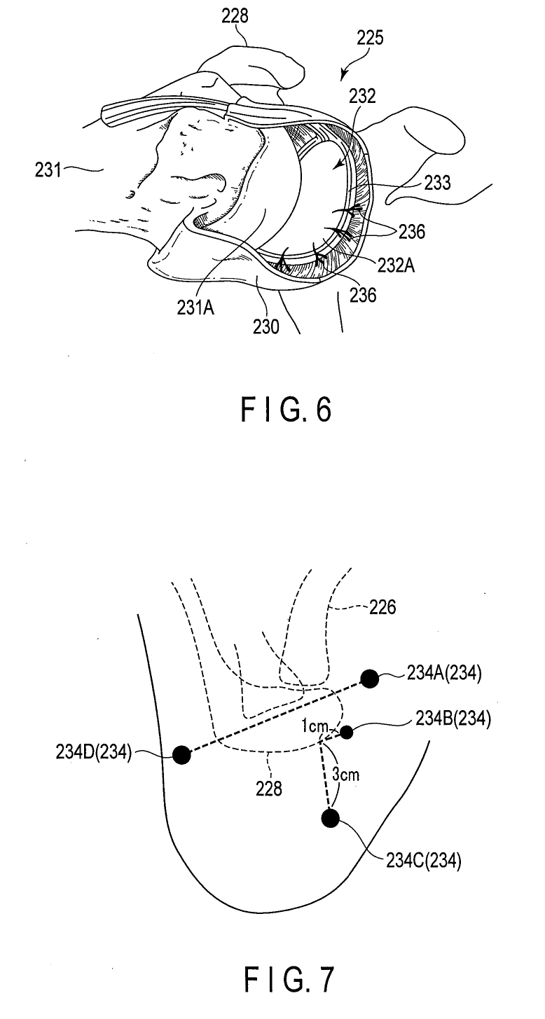

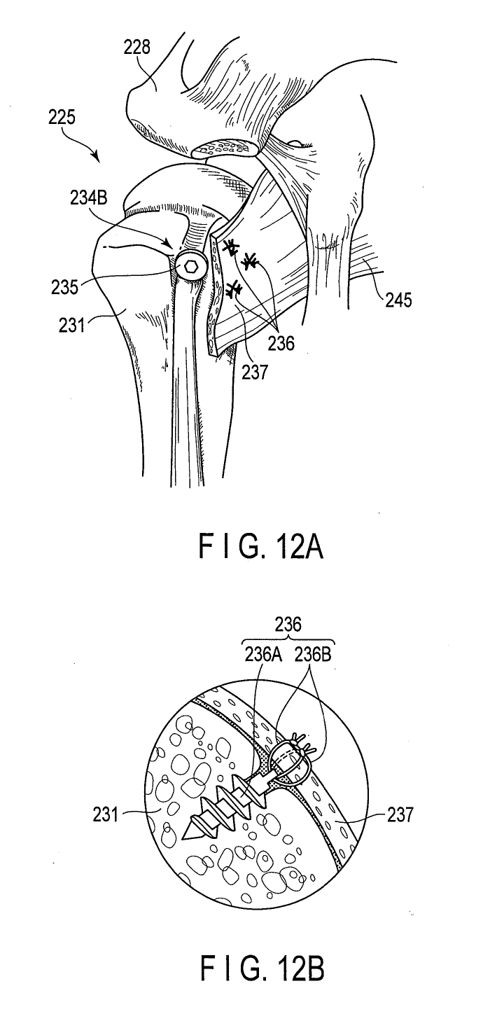

As shown in FIG. 4, the patient is fixed at, for example, a beach chair position. Needless to say, the patient may be fixed at a lateral recumbent position. FIG. 5 shows a position of a scapula upper arm joint 225 (the shoulder joint), and FIG. 6 shows an inner structure of the scapula upper arm joint 225 (the shoulder joint). FIG. 5 shows a clavicle 226, a scapula 227, an acromion 228, a humerus 231 and the like, and shows the scapula upper arm joint 225 at a position surrounded by a circle. In FIG. 6, a head 231A of the humerus 231 faces a labrum ligament complex 232. Their peripheries are covered with a ligament 230. FIG. 6 shows a state where a labrum 232A of the labrum ligament complex 232 is sutured at an anatomically correct position to a glenoid 233, and the treatment is completed. FIG. 7 shows portals 234 to approach the inside of the shoulder joint. The portals 234 include an anterior portal 234A, an anterosuperior portal 234B, a side portal 234C and a posterior portal 234D. A tubular cannula 235 or the like is inserted into each of the portals 234 (see FIG. 12A), so that the inside of the shoulder joint 200 can be accessed from the outside.

In the Bankart repair, the arthroscope 204 is inserted into the shoulder joint 200 via the posterior portal 234D. In addition, the ultrasonic probe 216 is inserted into one of the anterior portal 234A, the anterosuperior portal 234B and the side portal 234C to position this probe in the shoulder joint 200.

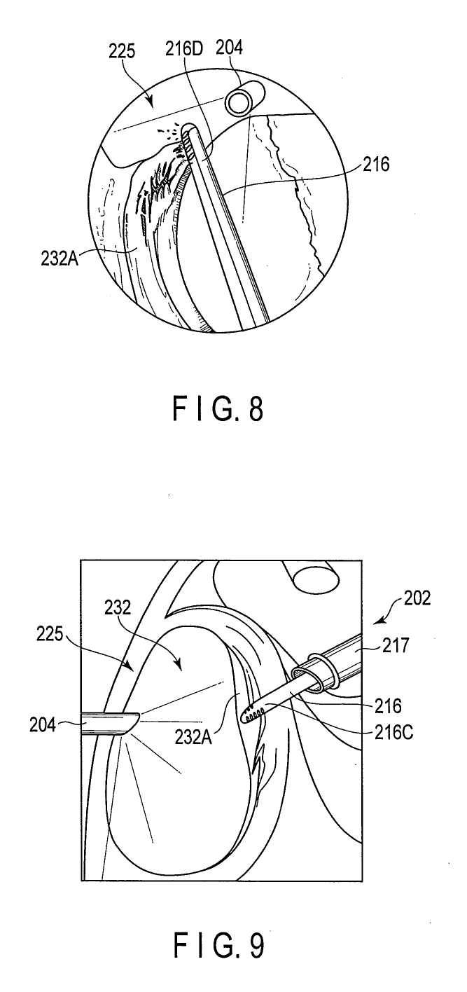

In a state seen with the arthroscope 204, the labrum ligament complex 232 of a damaged region is peeled from the glenoid 233 by use of the ultrasonic probe 216. In the present embodiment, as shown in FIG. 9, by using the cutting blades 224 of the side surfaces 216C of the ultrasonic probe 216 and the side surfaces 216C, the labrum 232A can be peeled from the glenoid 233. Additionally, as shown in FIG. 8, a torn region of the labrum ligament complex 232 is debrided by using the ultrasonically vibrated ultrasonic probe 216. The debridement can be performed by mainly using the cutting blades 224 of the upper surface 216B of the ultrasonic probe 216.

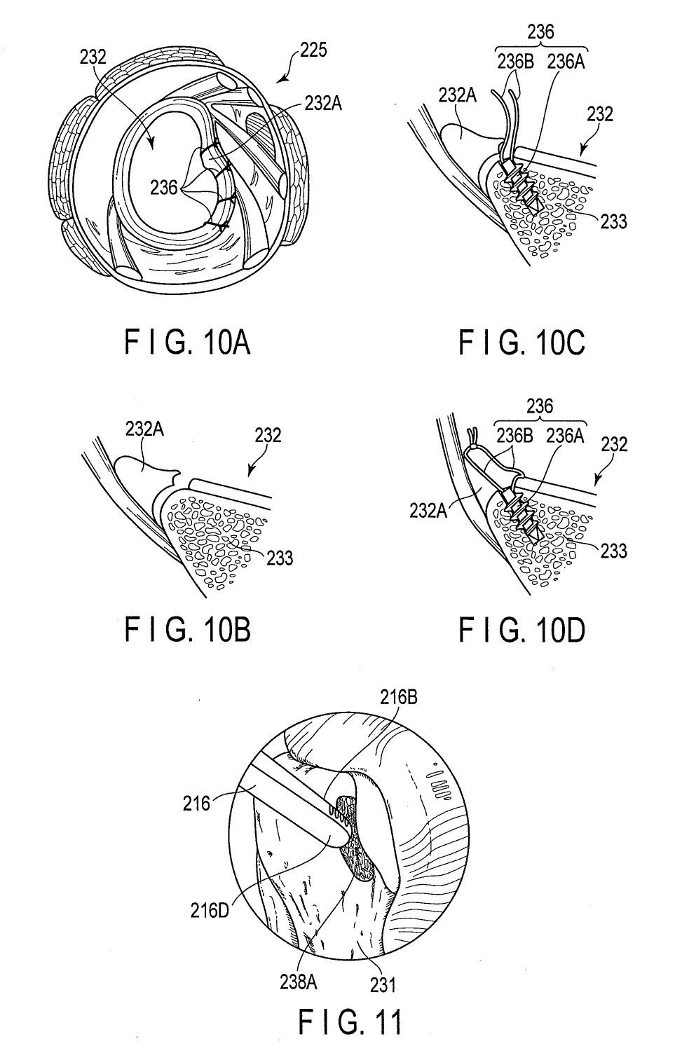

As shown in FIG. 10B, the labrum ligament complex 232 is peeled from the scapula glenoid 233, and as shown in FIG. 10C, an anchor 236A of anchors and sutures 236 is first fixed to a hard tissue (a bone or the like) on a glenoid 233 side. Subsequently, as shown in FIG. 10D, the labrum 232A is fixed to the scapula glenoid 233 by sutures 236B. Consequently, the labrum ligament complex 232 is disposed at its anatomically correct position. Additionally, although not shown, an articular capsule present around the complex can be sutured and contracted or fixed at its anatomically correct position by the anchors and sutures 236. As shown in FIG. 10A, the labrum 232A is fixed to regions by the anchors and sutures 236, and hence the labrum 232A is held at the correct position of the scapula glenoid 233. As described above, the Bankart repair is completed.

Next, shoulder rotator cuff repair will be described with reference to FIG. 11 to FIG. 18. The shoulder rotator cuff repair includes repair of a subscapular muscle tendon, repair of a supraspinatus tendon present under the acromion, removal of a subacromial bone spur (arthroscopic subacromial decompression) and the like, and the repair to be carried out varies depending on a condition of an affected area of the patient. The shoulder rotator cuff repair is carried out while circulating the perfusion liquid through the shoulder joint by the well-known method.

In the shoulder rotator cuff repair, the arthroscope 204 is inserted into the shoulder joint via, for example, the posterior portal 234D. Additionally, in the shoulder rotator cuff repair, the ultrasonic probe 216 is inserted into one of the anterior portal 234A, the anterosuperior portal 234B and the side portal 234C to position this probe in the shoulder joint.

First, it is assumed that the operator confirms tears in a subscapular muscle tendon 237 under the arthroscope 204. In this case, as shown in FIG. 11, a tendon remaining in a torn region (a humerus nodule footprint region 238A) of the subscapular muscle tendon 237 is debrided with the ultrasonic probe 216 which ultrasonically vibrates. In a state where the footprint region 238A is clean, the anchor 236A of the anchors and sutures 236 is fixed to the humerus 231 (see FIG. 12B). Further, as shown in FIG. 12A and FIG. 12B, the subscapular muscle tendon 237 is fixed to the humerus 231 by using the sutures 236B. It is to be noted that the fixing of the subscapular muscle tendon 237 by the anchors and sutures 236 may be performed together with fixing of a supraspinatus tendon 249 after a footprint region 238B of the supraspinatus tendon is cleaned and the bone spur 251 under the acromion 228 is removed as described later.

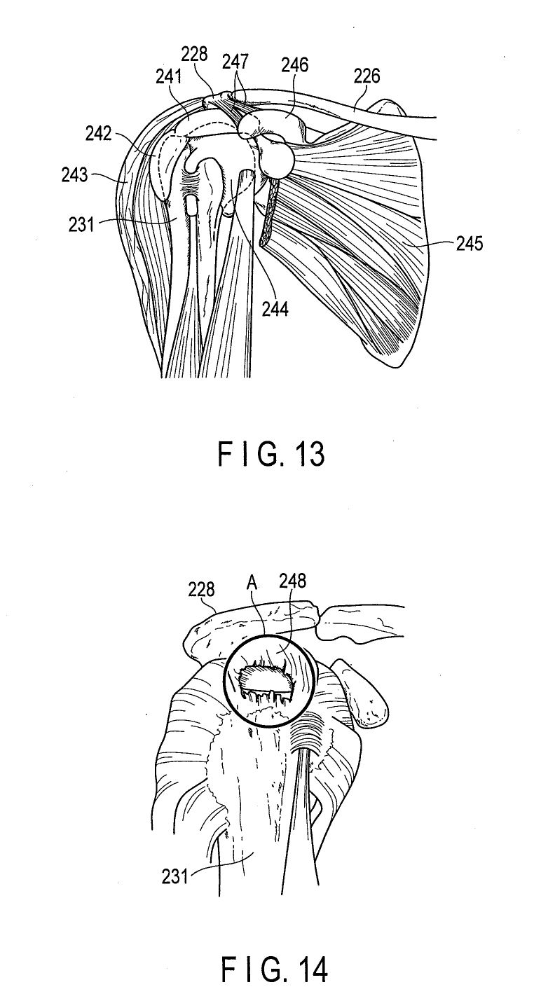

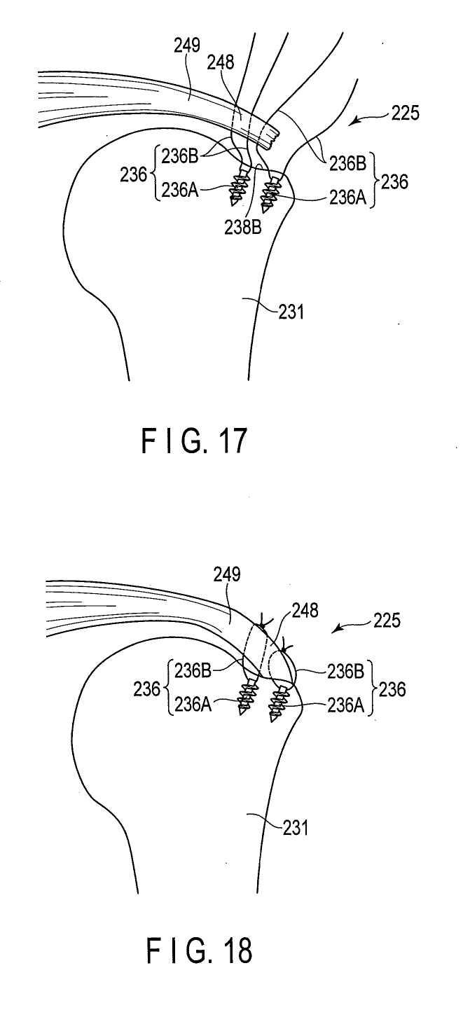

Prior to the repair of the supraspinatus tendon, the position of the bursa present in the shoulder joint will be described with reference to FIG. 13. On an upper side of the humerus 231 and a lower side of the acromion 228, a subacromial bursa 241 is positioned to cover the humerus 231, and a subdeltoid bursa 242 is adjacent to this subacromial bursa. Upper sides of these bursas are covered with a deltoid 243. An articular capsule 244 is adjacent to the subacromial bursa 241 and the subdeltoid bursa 242. A subscapular muscle 245 is connected to the humerus 231, and a coracoid process 246 is projected in the vicinity of the acromion 228. A coracoacromial ligament 247 is positioned along both of the coracoid process 246 and the acromion 228. Additionally, the humerus 231 is connected to the subscapular muscle tendon 237. In addition, the supraspinatus tendon 249 shown in FIG. 17 passes through a lower side of the acromion 228 to be fixed to the humerus 231.

Here, it is assumed that the operator confirms such a tear as shown by A of FIG. 14 in a cuff 248 of a region corresponding to the supraspinatus tendon 249 under the arthroscope 204. In this case, the ultrasonic probe 216 is first ultrasonically vibrated to remove the subacromial bursa 241 present around the cuff 248.

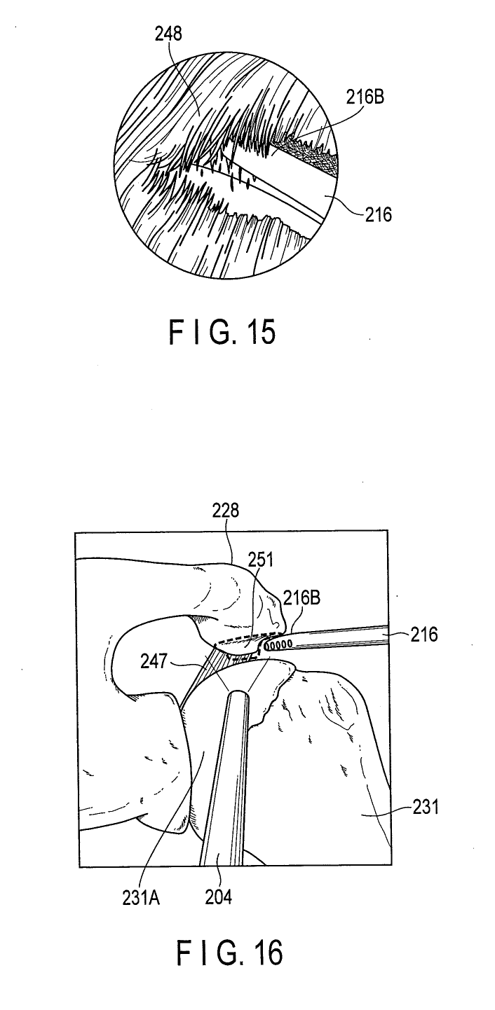

Further, the ultrasonic probe 216 is positioned in the vicinity of the cuff 248. As shown in FIG. 15, the ultrasonic probe 216 is ultrasonically vibrated to debride a periphery of the torn cuff 248. In addition, a tendon remaining at a position of a root of the supraspinatus tendon 249 (the humerus nodule footprint region 238B, see FIG. 17) is similarly debrided and cleaned by ultrasonically vibrating the ultrasonic probe 216. Additionally, the coracoacromial ligament 247 is excised by using the ultrasonically vibrated ultrasonic probe 216, and the cuff 248 of the region corresponding to the supraspinatus tendon 249 is sufficiently peeled from the humerus 231. It is to be noted that, even when a bursa such as the subacromial bursa 241 is removed for regeneration, there usually are not any problems.

Next, as shown in FIG. 16, the bone spur 251 present under the acromion 228 is removed by using the ultrasonic probe 216 which ultrasonically vibrates. In FIG. 16, a broken line shows a position of the bone spur 251. At this time, as to the ultrasonic probe 216, the ultrasonic probe 216 used in the abovementioned removal of the subacromial bursa 241, the cleaning of the cuff 248 and the like can be used as it is.

Thus, in the present embodiment, the removal of the subacromial bursa 241, the excision of the torn region of the cuff 248, the cleaning of the footprint region 238B of the cuff 248, the excision of the coracoacromial ligament 247 and the removal of the bone spur 251 under the acromion 228 can be performed by using the one ultrasonic probe 216.

As shown in FIG. 17, the anchor 236A of the anchors and sutures 236 is fixed to the footprint region 238B of the humerus 231. Further, as shown in FIG. 18, the cuff 248 is fixed to the footprint region 238B of the humerus 231 by use of the sutures 236B. As described above, the repair of the supraspinatus tendon 249 present under the acromion 228 is completed. Finally, the arthroscope 204, the ultrasonic probe 216, the cannula and the like are removed through the respective portals 234, and the articular capsule and the skin are sutured, to complete the shoulder rotator cuff repair.

According to the present embodiment, in a surgical treatment of the shoulder joint, the arthroscope 204 and the ultrasonic device are inserted into the shoulder joint, and the bursa is removed and the bone spur 251 present under the acromion 228 is removed in a state seen with the arthroscope 204 by use of the ultrasonic device which ultrasonically vibrates.

According to this method, the removal of the soft tissue of the bursa or the like and the removal of the hard tissue of the bone spur 251 or the like can be performed with one ultrasonic device, and hence it is not necessary to perform the treatment while replacing the devices, so that the surgical treatment can efficiently be performed and surgical treatment time can be shortened. In consequence, burdens on the patient can be decreased. Additionally, the probe of the ultrasonic device can be formed into a suitable shape, and the ultrasonic device can be formed to be smaller than a shaver or an abrader burr. Consequently, in the treatment in which the ultrasonic device is used, a movable range of the ultrasonic device can be increased, and a narrow region in a living body can easily be approached. Additionally, in the treatment of the ultrasonic device, a more precise and smoother treated surface can be formed than in a treatment in which the shaver or the abrader burr is used. In consequence, a patient's smooth joint movement can be realized.

Additionally, the abrader burr abrades the bone (the bone spur) that is the hard tissue by periaxial rotation, and hence loads that act on the abrader burr increase in a state where the bone is abraded. Consequently, the abrader burr might noticeably entirely be vibrated by the loads onto the treating portion. On the other hand, the ultrasonic device is not periaxially rotated but the bone can be resected only by moving (vibrating) the ultrasonic device in an axial direction. Consequently, the loads that act on the ultrasonic device during the use are small. In consequence, the ultrasonic device does not noticeably vibrate. That is, in the state where the bone is resected by the treating portion, leaping of the treating portion is not caused by rotary motion as in the abrader burr, and hence damages of a peripheral tissue can be decreased.

In addition, a surgeon uses the ultrasonic device and hence does not have to use a high frequency device. When the treatment is performed by using the high frequency device, there is the fear that the surface is invaded by heat. On the other hand, in the case where the ultrasonic device is used, a normal biological tissue is less invaded by heat, and thermal necrosis is prevented from being caused to the biological tissue.

According to the present embodiment, the arthroscope 204 and the ultrasonic device are inserted into the shoulder joint, and the footprint region 238B of the torn cuff 248 is debrided in the state seen with the arthroscope 204 by use of the ultrasonic device. According to this method, the footprint regions 238A and 238B can be debrided by using the ultrasonic device used in removing the bursa and the bone spur 251 as it is, and hence the surgical treatment can further efficiently be performed, so that the burdens on the patient can be decreased.

In addition, the probe of the ultrasonic device can be formed into the suitable shape, and the ultrasonic device can be formed to be smaller than the shaver or the abrader burr. Consequently, in the treatment in which the ultrasonic device is used, the movable range of the ultrasonic device can be increased, and the narrow region in the living body can easily be approached. Additionally, in the treatment of the ultrasonic device, the more precise and smoother treated surface can be formed than in the treatment in which the shaver or the abrader burr is used. In consequence, the patient's smooth joint movement can be realized.

Additionally, the abrader burr abrades the bone (the bone spur) that is the hard tissue by the periaxial rotation, and hence the loads that act on the abrader burr increase in the state where the bone is abraded. Consequently, the abrader burr might noticeably entirely be vibrated by the loads onto the treating portion. On the other hand, the ultrasonic device is not periaxially rotated but the bone can be resected only by moving (vibrating) the ultrasonic device in the axial direction. Consequently, the loads that act on the ultrasonic device during the use are small. In consequence, the ultrasonic device does not noticeably vibrate. That is, in the state where the bone is resected by the treating portion, the leaping of the treating portion is not caused by the rotary motion as in the abrader burr, and hence damages of the peripheral tissue can be decreased.

In addition, the surgeon uses the ultrasonic device and hence does not have to use the high frequency device. When the treatment is performed by using the high frequency device, there is the fear that the surface is invaded by heat. On the other hand, in the case where the ultrasonic device is used, the normal biological tissue is less invaded by heat, and the thermal necrosis is prevented from being caused to the biological tissue.

Additionally, according to the present embodiment, in the surgical treatment of the shoulder joint, the arthroscope 204 and the ultrasonic device are inserted into the shoulder joint, and the labrum ligament complex 232 is peeled from the scapula glenoid 233 and a damaged region of the labrum ligament complex 232 is debrided in the state seen with the arthroscope 204 by use of the ultrasonic device which ultrasonically vibrates.

According to this method, the peeling of the labrum ligament complex 232 and the debridement of the damaged region of the labrum ligament complex 232 can be performed with one ultrasonic device, and the treatment does not have to be performed while replacing the devices, so that the surgical treatment can efficiently be performed and the surgical treatment time can be shortened. In consequence, the burdens on the patient can be decreased.

In addition, the probe of the ultrasonic device can be formed into the suitable shape, and the ultrasonic device can be formed to be smaller than the shaver or the abrader burr. Consequently, in the treatment in which the ultrasonic device is used, the movable range of the ultrasonic device can be increased, and the narrow region in the living body can easily be approached. Additionally, in the treatment of the ultrasonic device, the more precise and smoother treated surface can be formed than in the treatment in which the shaver or the abrader burr is used. In consequence, the patient's smooth joint movement can be realized.

Additionally, the abrader burr abrades the bone (the bone spur) that is the hard tissue by the periaxial rotation, and hence the loads that act on the abrader burr increase in the state where the bone is abraded. Consequently, the abrader burr might noticeably entirely be vibrated by the loads onto the treating portion. On the other hand, the ultrasonic device is not periaxially rotated but the bone can be resected only by moving (vibrating) the ultrasonic device in the axial direction. Consequently, the loads that act on the ultrasonic device during the use are small. In consequence, the ultrasonic device does not noticeably vibrate. That is, in the state where the bone is resected by the treating portion, the leaping of the treating portion is not caused by the rotary motion as in the abrader burr, and hence the damages of the peripheral tissue can be decreased.

In addition, the surgeon uses the ultrasonic device and hence does not have to use the high frequency device. When the treatment is performed by using the high frequency device, there is the fear that the surface is invaded by heat. On the other hand, in the case where the ultrasonic device is used, the normal biological tissue is less invaded by heat, and the thermal necrosis is prevented from being caused to the biological tissue.

Furthermore, needless to say, it is possible to combine the Bankart repair with one or all of the treatments described in the shoulder rotator cuff repair, thereby constituting one surgical treatment.

What is claimed is:

1. A surgical treatment of a shoulder joint, comprising:
inserting an arthroscope and an ultrasonic device into the shoulder joint; and
removing a bursa and removing a bone spur present under an acromion in a state seen with the arthroscope by use of the ultrasonic device which ultrasonically vibrates.

2. The surgical treatment of the shoulder joint according to claim 1,
wherein the arthroscope and the ultrasonic device are inserted into the shoulder joint, and then a footprint region of a torn cuff is debrided in the state seen with the arthroscope by use of the ultrasonic device.

3. A surgical treatment of a shoulder joint, comprising:
inserting an arthroscope and an ultrasonic device into the shoulder joint; and
peeling a labrum ligament complex from a glenoid and debriding a damaged region of the labrum ligament complex in a state seen with the arthroscope by use of the ultrasonic device which ultrasonically vibrates.
